(19) United States
(12) Patent Application Publication
(10) Pub. No.: US 2024/0208951 A1
(43) Pub. Date: Jun. 27, 2024

(54) A PROCESS FOR PREPARING NAVAFENTEROL AND INTERMEDIATES THEREOF

(71) Applicant: GBR LABORATORIES PRIVATE LIMITED, HYDERABAD (IN)

(72) Inventors: Pratap Reddy Gaddam, Hyderabad (IN); Samhitha Reddy Gaddam, Hyderabad (IN); Madaalasa Reddy Gaddam, Hyderabad (IN); Udaya Kumar Reddy Mosali, Hyderabad (IN)

(73) Assignee: GBR LABORATORIES PRIVATE LIMITED, HYDERABAD (IN)

(21) Appl. No.: 18/276,831

(22) PCT Filed: Feb. 12, 2022
(86) PCT No.: PCT/IN2022/050122

§ 371 Date: Aug. 10, 2023

(30) Foreign Application Priority Data
Feb. 12, 2021 (IN) 202141004550

(51) Int. Cl. C07D 409/14

(52) U.S. Cl. CPC C07D 409/14

(57) ABSTRACT

The present invention relates to a process for preparation of Navafenterol of Formula (I) and intermediate compounds of Formula (VII) and Formula (XIII) by chiral sulfide mediated epoxidation. The process involves preparation of the intermediate compounds of Formula (VII) and Formula (XIII) followed by preparation of Navafenterol from the compound of Formula (XIII) either by condensation with the compound of Formula (XVI); or via intermediates of the compound of Formula (XVIII) and compound of Formula (XV); or via intermediates of the compound of Formula (XI-a) and compound of Formula (XVI). The process uses simple raw materials and reagents. Further, the process is cost effective and gives higher yield and better purity.

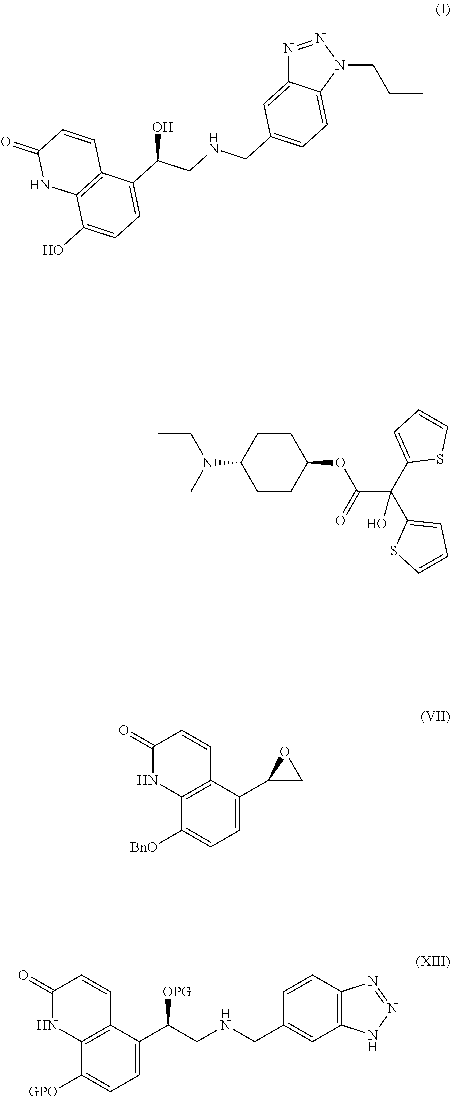

### CROSS REFERENCE TO RELATED APPLICATION

This application is a national phase entry of PCT Application No PCT/IN2022/050122, filed Feb. 12, 2022, titled “A PROCESS FOR PREPARING NAVAFENTEROL AND INTERMEDIATES THEREOF” which claims priority to Indian Application No. 202141004550, filed Feb. 12, 2021, titled “A PROCESS FOR PREPARING NAVAFENTEROL AND INTERMEDIATES THEREOF” which are incorporated herein by reference in their entirety.

### FIELD OF THE INVENTION

The present invention relates to a process for preparation of chiral intermediates of bi-functional muscarinic β2-agonist (MABAs) and more particularly to a process for preparation of chiral intermediates of Navafenterol by chiral sulfide mediated epoxidation.

### BACKGROUND OF THE INVENTION

Respiratory disorders are one of the leading causes of death in the world. Respiratory disorders are associated mainly with tobacco smoking, air pollution or occupational exposure, which can cause obstruction of airflow in the lungs resulting in bouts of breathlessness. COPD, bronchial asthma, chronic bronchitis, asthmatic bronchitis and emphysema are some of the respiratory disorders.

Bronchodilators are frequently used to treat respiratory disorders. The bronchodilators help loosen tight muscles of the airways leading to the widening of airways. The widening of airways in turn leads to easy breathing. Various class of compounds work as bronchodilators such as the β-adrenoceptor agonists, muscarinic receptor antagonists and the like. These bronchodilators are available in both short acting and long-acting forms.

Of particular interest are the long-acting dual-pharmacology muscarinic antagonist/β2-adrenoceptor agonists (MABA). The MABAs function by combining muscarinic antagonism and β2-agonism in a single molecule. Navafenterol is one such muscarinic antagonist/β2-adrenoceptor agonist, and it is currently under development as a long-acting bronchodilator drug. Due to the dual pharmacological activity, it is anticipated that Navafenterol would offer greater efficacy than single-mechanism long-acting muscarinic antagonists (LAMAs) or long-acting β2 receptor agonists (LABAs).

There is a growing interest in the development of cost effective and environmentally friendly processes for muscarinic antagonist/β2-adrenoceptor agonists (MABA). The compound Navafenterol can be derived from a class of compounds called Quinolinones. The patent U.S. Pat. No. 7,521,558B2 by Theravance Inc discloses a crystalline form of biphenyl compound, and a process of preparing the compound involving formation of the intermediates 2-Quinolinones. The patent application WO2006122788A1 by Almirall Prodesfarma SA et al describes 4-(2-amino-1-hydroxyethyl) phenol derivatives as β2 adrenergic agonists involving formation of intermediates 2-Quinolinones. These are some of the known processes for the preparation of some agonists and their intermediates. However, the known processes involving the preparation of intermediates are expensive and have extended production time. There is a need for a process of preparation of chiral intermediates of Navafenterol which is cost effective, less time consuming and has fewer steps of synthesis. There is a further need of a process that avoids the usage of toxic reagents like borane derivative during the (chiral) selective reduction.

Further, there is a need for an industrially feasible process for preparation of Navafenterol and its chiral intermediates with improved yield and purity, thereby reducing production cost and time. Also, there is a need to synthesize Navafenterol from simple raw materials using safe and simple process.

### SUMMARY OF THE INVENTION

The present invention describes a process for preparation of Navafenterol compound having the Formula (I) and the process for the preparation of chiral intermediate compounds of Formula I.

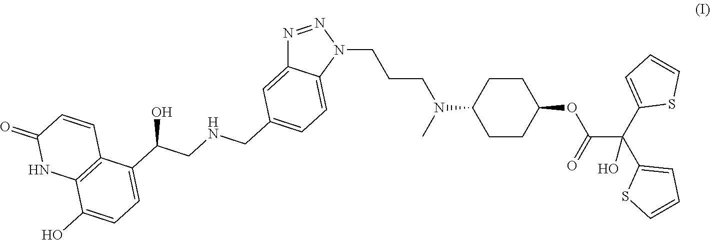 [SMILES: CN(CCCn1nnc2cc(CNC[C@H](O)c3ccc(O)c4[nH]c(=O)ccc34)ccc21)[C@H]1CC[C@H](OC(=O)C(O)(c2cccs2)c2cccs2)CC1]

The process includes the steps of addition of the compound of Formula (II) to a solvent followed by adding a base and a benzylating agent to obtain a compound of Formula (III); followed by addition of the compound of Formula (III) to an acid and a brominating agent to make a reaction mixture; to obtain a compound of Formula (IV); carbonylation of compound of formula IV with strong base to obtain compound of formula V; chiral epoxidation of compound of formula V using chiral sulphide (VI), in presence of base to obtain compound of formula VII; cleavage of chiral epoxide compound of formula VII using brominating agent to obtain compound of formula VIII; treatment of compound of formula VIII with compound of formula IX in base to obtain compound of formula XI; hydrogenation of compound of the Formula XI in presence of catalyst to obtain compound of Formula XII, protection of compound of Formula XII, with a protecting agent to obtain compound of Formula XIII; followed by synthesis of Navafenterol having the Formula (I) from the compound of Formula (XIII)
(1) by condensation with the compound of Formula (XVI); or
(2) via intermediates of the compound of Formula (XVIII) and compound of Formula (XV); or
(3) via intermediates of the compound of Formula (XI-a) and compound of Formula (XVI).

The process of synthesis of Navafenterol from the intermediate compound of Formula (XIII) and compound of Formula (XVI) includes the steps of condensation of compound of Formula XIV by adding a solution of compound of Formula XV to obtain compound of Formula XVI; and condensation of compound of formula XIII and compound of formula XVI in presence of base to obtain Navafenterol of Formula I.

The process of synthesis of Navafenterol from the intermediate compound of Formula (XIII) via intermediates of the compound of Formula (XVIII) and compound of Formula (XV) includes the steps of condensation of compound of Formula (XIII) with compound of Formula (XVII) to give compound of Formula (XVIII) and condensation of compound of Formula (XVIII) with compound of Formula (XV) to give compound of Formula (I).

The process of synthesis of Navafenterol from the intermediate compound of Formula (XIII) via intermediates of the compound of Formula (XI-a) and compound of Formula (XVI) includes the steps of protection of compound of formula VIII with protecting agents to obtain compound of formula VIII(a); treatment of compound of formula VIII(a) with compound of formula IX(a) to obtain compound of formula XI(a); condensation of compound of Formula XI(a) and compound of Formula XVI to obtain compound of formula XIX; hydrogenation of compound of formula XIX to obtain compound of formula I.

In the process for the preparation of the intermediate compound of Formula XIII, the addition of compound of Formula II is carried out in presence of solvents selected from acetone, THF or DMF and base selected from group of K2CO3, Na2CO3, or Cs2CO3; followed by addition of benzylating agent selected from benzyl bromide, benzyl chloride or benzyl iodide to obtain compound of formula III; addition of compound of Formula III is carried out in halosolvents, acetic acid, formic acid, hydrobromic acid; further bromination with bromine, 1,3-dibromohydantoin, tetra butyl ammonium tribromide, NBS to obtain compound of formula IV; carbonylation of compound of formula IV is carried out in presence of solvents selected from tetrahydrofuran, methyl tert-butylether, diisopropyl ether, or diethyl ether, followed by treating with strong base selected from n-butyl lithium, s-butyl lithium, lithium diisopropylamide, potassium bis(trimethylsilyl)amide, in turn addition of N,N-dimethylformamide and tetrahydrofuran in the ratio of 1:1 to obtain compound of formula V; chiral epoxidation of compound of formula V is carried out using chiral sulphide derivative (VI), in presence of base selected from potassium hydroxide, sodium hydroxide, lithium hydroxide, in tert-butyl alcohol, isopropyl alcohol, methanol to obtain compound of formula VII; cleavage of chiral epoxide compound of the formula VII is carried out in solvents selected from Tetrahydrofuran, or halo solvents, and the brominating agent selected from bromine, 1,3-dibromohydantoin, NBS to obtain compound of formula VIII; treatment of compound of formula VIII with compound of formula IX is carried out in solvents selected from acetone, THF, DMF using base selected from K2CO3, Na2CO3, Cs2CO3, to obtain compound of formula XI; hydrogenation of compound of formula XI is carried out in methanol, ethanol, Isopropyl alcohol, ethyl acetate, in presence of catalyst to obtain compound of formula XII; and protection of compound of formula XII is carried out with protecting agents selected from THP, TBDMS, TMS and benzyl bromide, benzyl chloride, benzyl iodide, in solvents selected from acetone, THF, DMF, acetonitrile, 2-metyl THF, MIBK, halo solvents using base selected from K2CO3, Na2CO3, Cs2CO3, imidazole to obtain formula XIII. The addition of benzylating agent is carried out at the temperature of about 0° C. to 5° C.; carbonylation of compound of formula IV is carried out at the temperature of about 0° C. to −78° C. for 2 to 3 hours, and cleavage of chiral epoxide of formula VII is carried out at the temperature of about 10° C.-15° C.; hydrogenation is carried out in presence of catalyst selected from the group of Pd/C, Pd/BaSO4, or Raney nickel.

In the process for the preparation of Navafenterol of the Formula I from the compound of Formula (XIII) by condensation with the compound of Formula (XVI); the condensation of Formula XIV is carried out in solvent toluene using base sodium methoxide followed by adding a solution of compound of Formula XV in toluene to obtain compound of Formula (XVI); isolation of compound of formula XVI is carried out in toluene under cooling; condensation of compound of Formula XIII and compound of Formula XVI is carried out in presence of base TEA in toluene; the isolation of formula I in toluene is carried out under cooling. The condensation of compound of Formula XIII and compound of Formula XVI is carried out at the temperature at below 10° C.-15° C. for at least 4 hours; the condensation of compound of Formula XVIII in solvent toluene using base sodium methoxide is carried out at the temperature at about 75° C. and 85° C.

In the process for the preparation of Navafenterol of the Formula I from the compound of Formula (XIII) via intermediates of the compound of Formula (XVIII) and compound of Formula (XV), the reaction of compound of Formula XIII with compound of Formula XVII in presence of base TEA is carried out under cooling in dichloromethane to obtain compound of XVIII; isolation of compound of formula XVIII as residue; condensation of compound of Formula XVIII is carried out in solvent toluene using base sodium methoxide followed by adding a solution of Formula XV in toluene to obtain compound of Formula I; isolation of compound of formula I is carried out in toluene under cooling.

In the process for the preparation of Navafenterol of the Formula I from the compound of Formula (XIII) via intermediates of the compound of Formula (XI-a) and compound of Formula (XVI), the protection of compound of formula VIII is carried out with protecting agents selected from THP, TBDMS, TMS and benzyl bromide, benzyl chloride, benzyl iodide, in solvents selected from acetone, THF, DMF, acetonitrile, 2-metyl THF, MIBK, using base selected from K2CO3, Na2CO3, Cs2CO3, imidazole to obtain compound of formula VIII(a); treatment of compound of formula VIII(a) with compound of formula IX (a) in carried out in solvents selected from acetone, THF, DMF using base selected from K2CO3, Na2CO3, Cs2CO3, to obtain compound of formula XI(a); condensation of compound of Formula XI (a) and compound of Formula XVI is carried out in presence of base TEA in toluene, and isolation is carried out in toluene under cooling to obtain compound of formula XIX; hydrogenation of compound of formula XIX is carried out in acidic medium using solvents selected from methanol, ethanol, Isopropyl alcohol, ethyl acetate, in presence of catalyst selected from the group of Pd/C, Pd/BaSO4, or Raney nickel to obtain compound of formula I.

### DETAILED DESCRIPTION OF THE INVENTION

The foregoing objects of the present invention are accomplished and the problems and shortcomings associated with the prior art, techniques and approaches are overcome by the present invention as described below in the preferred embodiments.

All materials used herein were commercially purchased as described herein or prepared from commercially purchased materials as described herein.

Although specific terms are used in the following description for sake of clarity, these terms are intended to refer only to particular structure of the invention selected for illustration in the drawings and are not intended to define or limit the scope of the invention.

References in the specification to “preferred embodiment” means that a particular feature, structure, characteristic, or function described in detail thereby omitting known constructions and functions for clear description of the present invention.

In one aspect, the present invention relates to a process for preparation of Navafenterol having the below given formula (I).

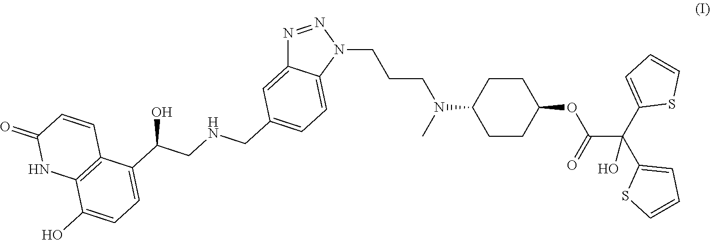 [SMILES: CN(CCCn1nnc2cc(CNC[C@H](O)c3ccc(O)c4[nH]c(=O)ccc34)ccc21)[C@H]1CC[C@H](OC(=O)C(O)(c2cccs2)c2cccs2)CC1]

In another aspect, the present invention relates to a process for the preparation of chiral intermediate compounds of the Formula (VII) and (XIII) of Navafenterol.

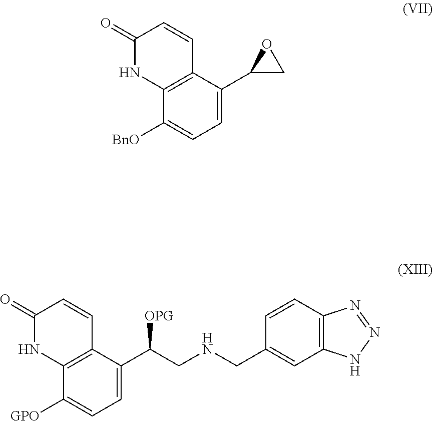

In an embodiment, the process of preparation of the chiral intermediate compound of the Formula (VII) includes the steps of:

a) addition of the compound of Formula (II) to a solvent followed by adding a base and a benzylating agent to obtain a compound of Formula (III);

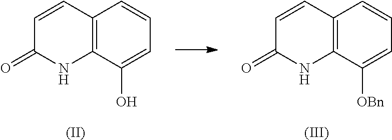 [SMILES: O=c1ccc2cccc(O)c2[nH]1 ; O=c1ccc2cccc(OCc3ccccc3)c2[nH]1 ; "(II)" ; "(III)"]

b) addition of the compound of Formula (III) to an acid and a brominating agent to make a reaction mixture; to obtain a compound of Formula (IV);

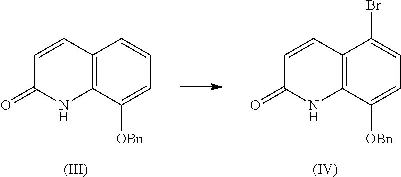 [SMILES: O=c1ccc2cccc(OCc3ccccc3)c2[nH]1 ; O=c1ccc2c(Br)ccc(OCc3ccccc3)c2[nH]1 ; "(III)" ; "(IV)"]

c) carbonylation of compound of formula IV with strong base to obtain the compound of formula V;

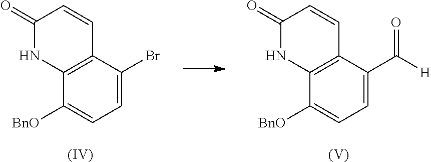

d) chiral epoxidation of compound of formula V using chiral sulphide (VI), in presence of base to obtain compound of formula VII;

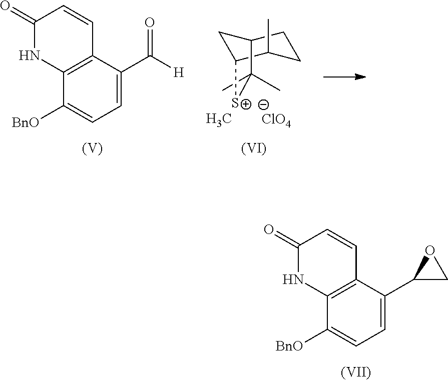

In this embodiment, for synthesis of the compound of the Formula (VII), the process is described in detailed herein:

a) the addition of compound of Formula II is carried out in presence of solvents selected from acetone, THF or DMF and base selected from group of K₂CO₃, Na₂CO₃, or Cs₂CO₃; followed by addition of benzylating agent selected from benzyl bromide, benzyl chloride or benzyl iodide to obtain compound of formula III. The addition of benzylating agent is carried out at the temperature of about 0° C. to 5° C.
b) the addition of compound of formula III is carried out in halosolvents, acetic acid, formic acid, hydrobromic acid; further the bromination is carried out with brominating agents selected from bromine, 1,3-dibromohydantoin, tetra butyl ammonium tribromide, NBS to obtain compound of formula IV.
c) the carbonylation of compound of formula IV is carried out in presence of solvents selected from tetrahydrofuran, methyl tert-butylether, diisopropyl ether, or diethyl ether, followed by treating with strong base selected from n-butyl lithium, s-butyl lithium, lithium diisopropylamide, potassium bis(trimethylsilyl)amide, in turn addition of N,N-dimethylformamide and tetrahydrofuran in the ratio of 1:1 to obtain compound of formula V. The carbonylation of formula IV is carried out at the temperature of about 0° C. to −78° C. for 2 to 3 hours.
d) the chiral epoxidation of compound of formula V using chiral sulphide derivative (VI), is carried out in presence of base selected from potassium hydroxide, sodium hydroxide, lithium hydroxide, in solvents selected from tert-butyl alcohol, isopropyl alcohol, methanol to obtain compound of formula VII.

In a further embodiment, the process of preparation of the chiral intermediate compound of the Formula (XIII) from the intermediate compound of the Formula (VII) includes the steps of:

a) cleavage of chiral epoxide of compound of formula VII using brominating agent to obtain compound of formula VIII;

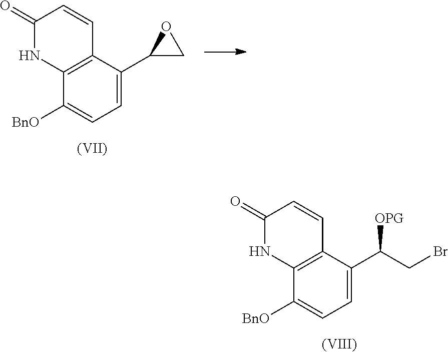

b) treatment of compound of formula VIII with compound of formula IX in base to obtain compound of formula XI;

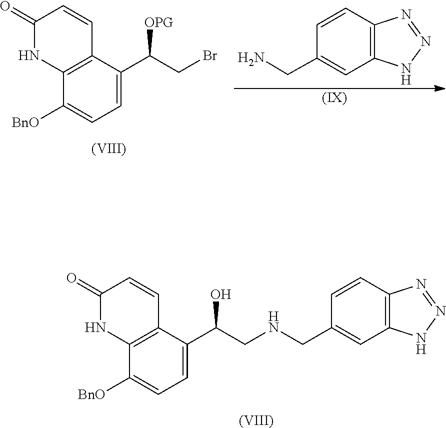

c) hydrogenation of the compound of Formula XI in presence of catalyst to obtain compound of Formula XII,

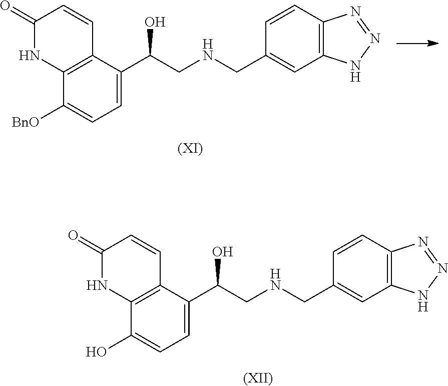

d) protection of compound of Formula XII, with a protecting agent to obtain compound of Formula XIII;

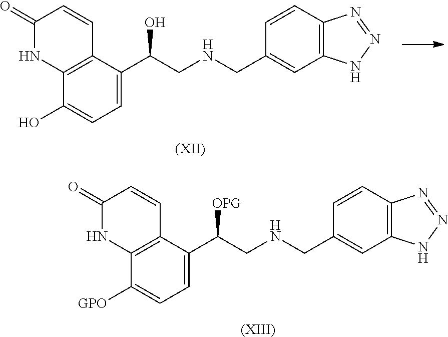

In this embodiment, for synthesis of the compound of the Formula (XIII) from the compound of Formula (VII), the detailed process is described in detailed herein:

a) the cleavage of chiral epoxide compound of the formula VII is carried out in solvents selected from Tetrahydrofuran, or halo solvents, and the brominating agent selected from bromine, 1,3-dibromohydantoin, NBS to obtain compound of formula VIII. The cleavage of chiral epoxide of formula VII is carried out at the temperature of about 10° C.-15° C.
b) the treatment of compound of formula VIII with compound of formula IX in solvents selected from acetone, THF, DMF using base selected from K₂CO₃, Na₂CO₃, Cs₂CO₃, to obtain compound of formula XI.
c) the hydrogenation of compound of formula XI in methanol, ethanol, Isopropyl alcohol, ethyl acetate, in presence of catalyst to obtain compound of formula XII. The hydrogenation is carried out in presence of catalyst selected from the group of Pd/C, Pd/BaSO₄, or Raney nickel.
d) the protection of compound of formula XII with protecting agents selected from THP, TBDMS, TMS and benzyl bromide, benzyl chloride, benzyl iodide, in solvents selected from acetone, THF, DMF, acetonitrile, 2-metyl THF, MIBK, halo solvents using base selected from K₂CO₃, Na₂CO₃, Cs₂CO₃, imidazole to obtain compound of formula XIII.

The compound Navafenterol having the formula I is synthesized by various routes as described below:

The Route 1 of synthesis of compound of the Formula (I) from the intermediate compound of Formula (XIII) and compound of Formula (XVI) includes the steps of:

a) condensation of compound of Formula XIV by adding a solution of compound of Formula XV to obtain compound of Formula XVI; and

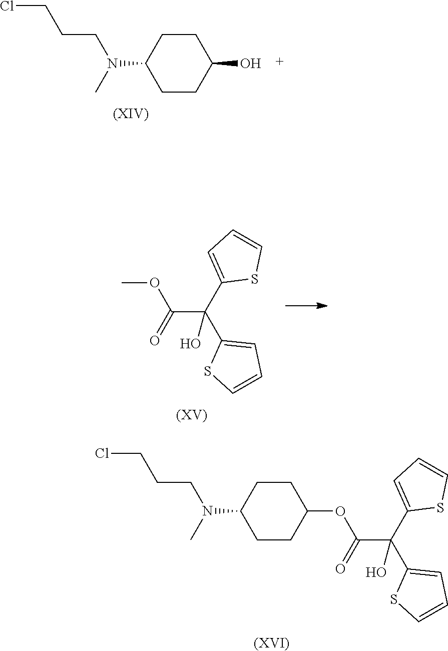

b) condensation of compound of formula XIII and compound of formula XVI in presence of base to obtain Navafenterol of Formula I.

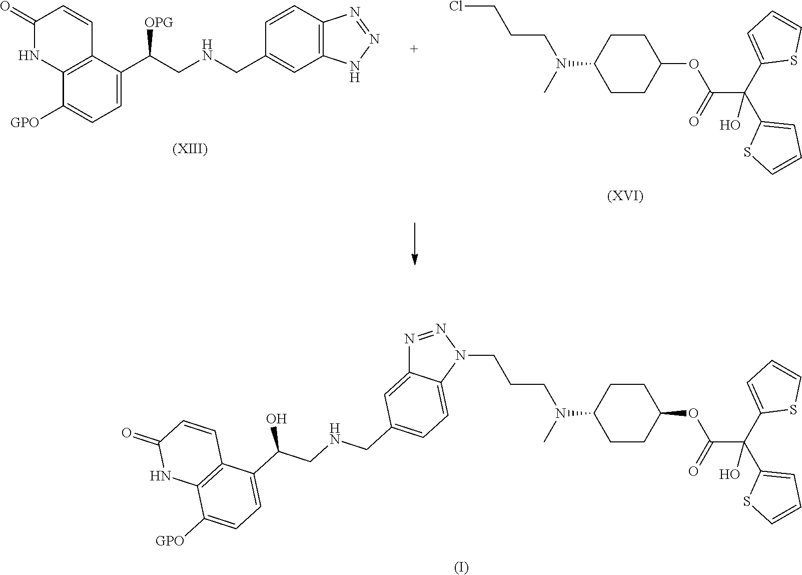

In this embodiment, for synthesis of the compound of the Formula (I), the detailed process is described in detailed herein:

a) the condensation of compound of Formula XIV is carried out in solvent toluene using base sodium methoxide followed by adding a solution of compound of Formula XV in toluene to obtain compound of Formula (XVI);
b) the isolation of compound of formula XVI is carried out in toluene under cooling;
c) the condensation of compound of Formula XIII and compound of Formula XVI in carried out in presence of base TEA in toluene;
d) the isolation of compound of formula I in toluene under cooling.

The Route 2 of synthesis of compound of the Formula (I) from the intermediate compound (XVIII) and compound (XV) includes the steps of:

a) condensation of compound of Formula (XIII) with compound of Formula (XVII) to give compound of Formula (XVIII).

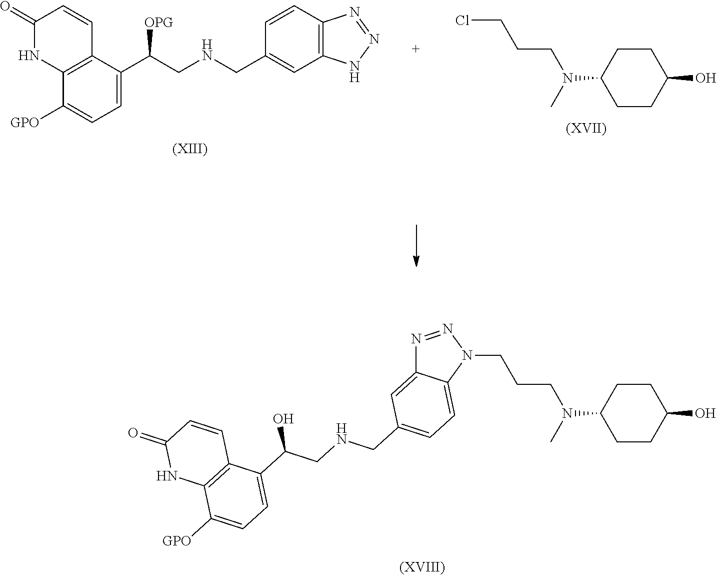

b) condensation of compound of Formula (XVIII) with compound of Formula (XV) to give compound of Formula (I).

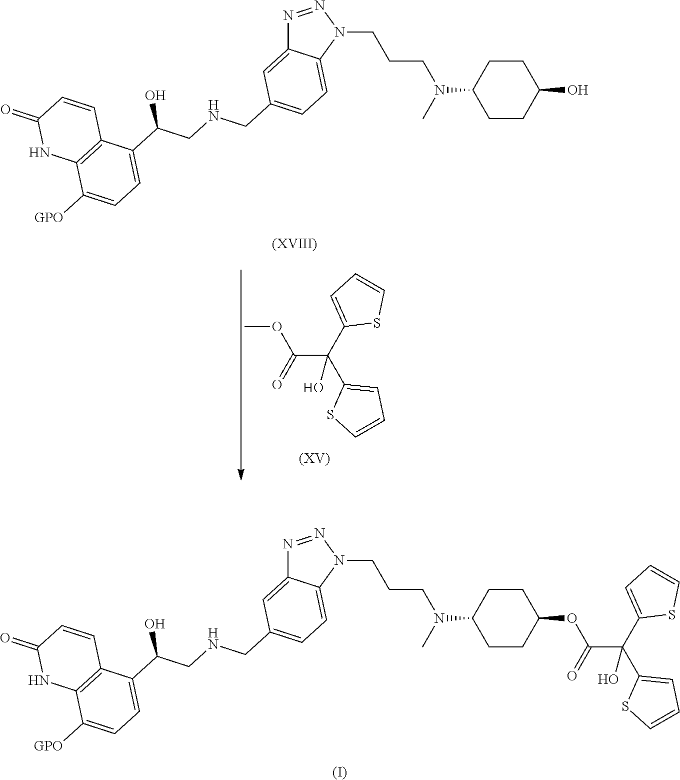

In this embodiment, for synthesis of the compound of the Formula (I), the detailed process is described in detailed herein:

a) reaction of compound of Formula XIII with Formula XVII in presence of base TEA under cooling is carried out in dichloromethane to obtain compound of XVIII; condensation of Formula XIII and Formula XVII is carried out at the temperature at below 10° C.-15° C. for at least 4 hours.
b) isolation of compound of formula XVIII as residue is carried out.
c) condensation of compound of Formula XVIII is carried out in solvent toluene using base sodium methoxide followed by adding a solution of compound of Formula XV in toluene to obtain compound of Formula I. The condensation of compound of Formula XVIII in solvent toluene using base sodium methoxide is carried out at the temperature at about 75° C. and 85° C.
d) isolation of compound of formula I is carried out in toluene under cooling.

The Route 3 of synthesis of compound of the Formula (I) from the intermediate compound of Formula (XI-a) and compound of Formula (XVI) includes the steps of:

a) protection of compound of formula VIII with protecting agents to obtain compound of formula VIII (a);

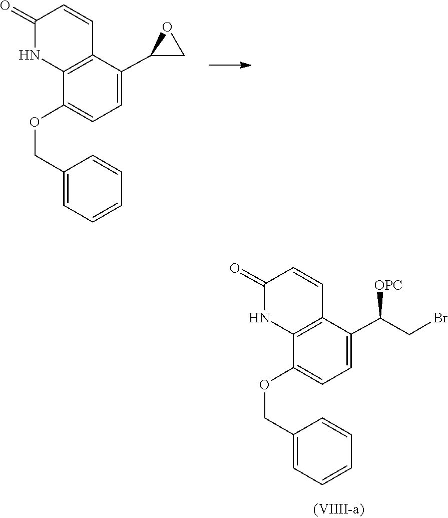

b) treatment of compound of formula VIII (a) with compound of formula IX (a) to obtain compound of formula XI (a);

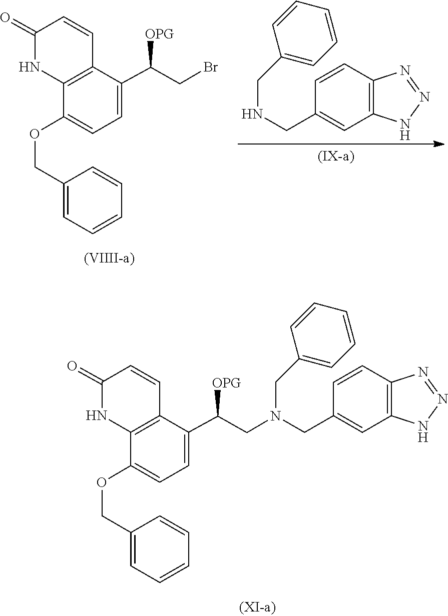

c) condensation of compound of Formula XI (a) and compound of Formula XVI to obtain compound of formula XIX;

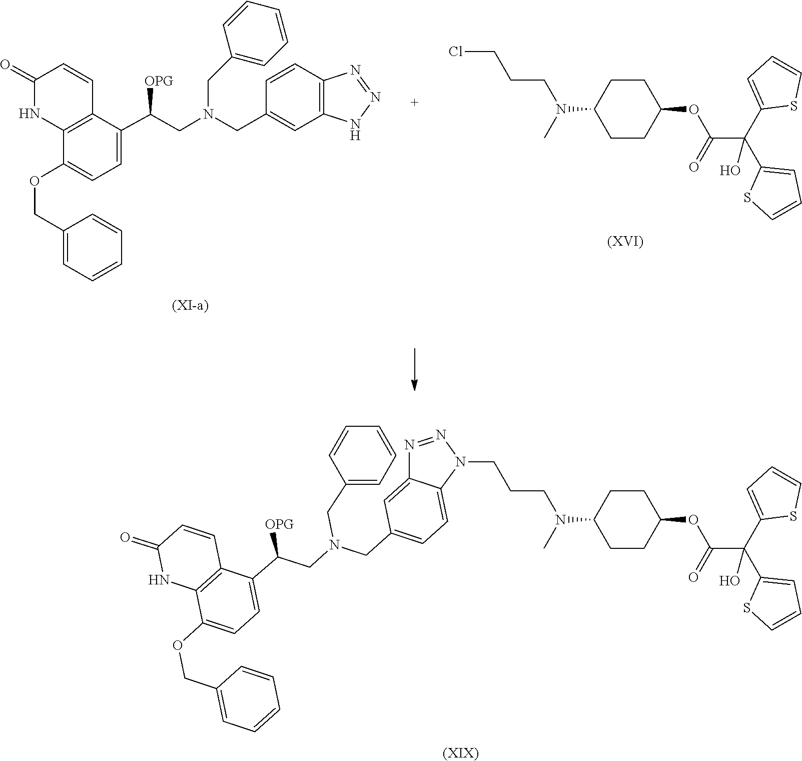

d) hydrogenation of compound of formula XIX to obtain compound of formula I.

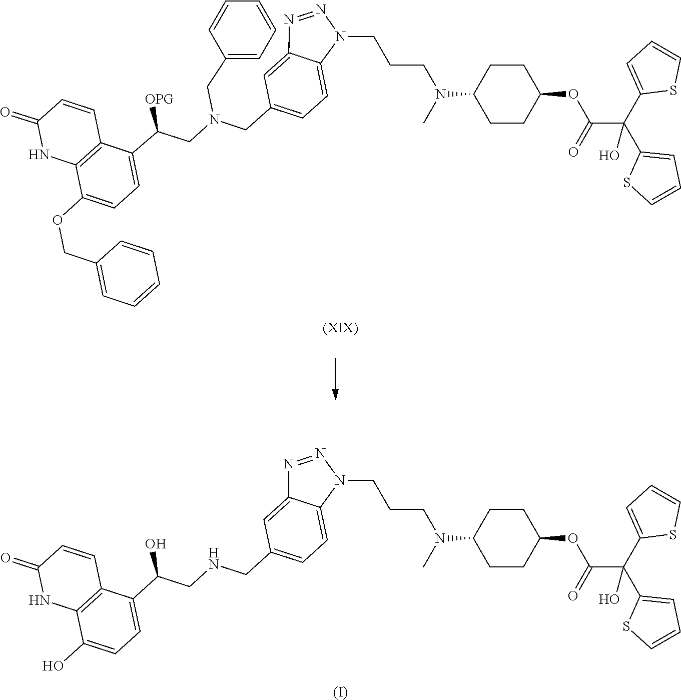

In this embodiment, for synthesis of the compound of the Formula (I), the detailed process is described in detailed herein:

a) protection of compound of formula VIII is carried out with protecting agents selected from THP, TBDMS, TMS and benzyl bromide, benzyl chloride, benzyl iodide, in acetone, THF, DMF, acetonitrile, 2-metyl THF, MIBK, using base selected from K₂CO₃, Na₂CO₃, Cs₂CO₃, imidazole to obtain compound of formula VIII (a);b) treatment of compound of formula VIII (a) with compound of formula IX (a) is carried out in solvents selected from acetone, THF, DMF using base selected from K₂CO₃, Na₂CO₃, Cs₂CO₃, to obtain compound of formula XI (a);
c) condensation of compound of Formula XI (a) and compound of Formula XVI is carried out in presence of base TEA in toluene, and isolation in toluene under cooling to obtain compound of formula XIX;
d) hydrogenation of compound of formula XIX in acidic medium is carried out using the solvents selected from methanol, ethanol, Isopropyl alcohol, ethyl acetate, in presence of catalyst selected from the group of Pd/C, Pd/BaSO₄, or Raney nickel to obtain compound of formula I.

These and other embodiments will be apparent to those of skill in the art and others in view of the following detailed description of some embodiments. It should be understood, however, that this summary, and the detailed description illustrate only some examples of various embodiments, and are not intended to be limiting to the invention as claimed.

The process of the present invention uses simple raw materials and reagents. Advantageously, the process of the present invention is cost effective and results in high yield of the end product with maximum purity. Further, the process is simple and uses safer process operations. It leads to reduction in the overall production time.

### EXAMPLES

Only a few examples and implementations are disclosed. Variations, modifications, and enhancements to the described examples and implementations and other implementations can be made based on what is disclosed.

Examples are set forth herein below and are illustrative of different amounts and types of reactants and reaction conditions that can be utilized in practicing the disclosure. It will be apparent, however, that the disclosure can be practiced with other amounts and types of reactants and reaction conditions than those used in the examples, and the resulting devices various different properties and uses in accordance with the disclosure above and as pointed out hereinafter.

### Example 1: Preparation of the Chiral Intermediate Compound of the Formula (VII)

1. To a solution of 8-Hydroxyquinolin-2-(1H)-one (II) (5.0 g, 0.031 mol) in acetone (40 mL), K₂CO₃ (5.1 g, 0.037 mol) and benzyl bromide (4.4 mL, 0.037 mol) were added at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂SO₃ solution and then extracted with EtOAc (3×40 mL). The combined organic layers were washed with sat. NaHCO₃ solution followed by water and brine solution and dried over Na₂SO₄. The solvent was evaporated and the resulting residue was purified by flash chromatography to afford the compound (IV) (80% yield) as a colourless solid.
3. The above bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C.for 2 h and then treated with dry N,N-dimethylformamide (12.7 mL, 0.17 mol) as a 1:1 solution in dry THF. The resulting mixture was stirred at −78° C.for 0.75 h and warmed slowly up to room temperature. Then the mixture was diluted with diethyl ether, washed with water followed by a brine solution and dried over MgSO₄. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc-hexane as an eluent gave the epoxide (VII) in 80% yield.

### Example 2: Preparation of the Chiral Intermediate Compound of the Formula (XIII)

1. A stirred solution of (VII) in THF was cooled to 10-15° C. and aq.HBr (48% 1.1 m.eq) was added. The resulting mixture was stirred at the same temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate was distilled under vacuum at below 45° C. to get the compound bromo hydrin (VIII).
2. To a stirred solution of THF (25 mL) and bromo hydrin (VIII) (2.5 g, 0.0085 mol) a solution of (1H-benzo[d] [1,2,3] triazol-6-yl) methanamine (IX) (0.92 g, 0.0069 mol) in THF 25 ml was added in a dropwise manner at room temperature under nitrogen. After complete addition, the mixture was allowed to warm to 50° C. and stirred for 4 h. The resulting homogeneous mixture was quenched with saturated aqueous Na₂SO₃ and extracted with CH₂Cl₂ and purified by flash chromatography on silica gel using a gradient mixture of ethyl acetate-n-hexane to give the compound (XI) (72% yield) as a pale-yellow solid.
3. To the above compound (XI) (1.5 g, 0.0035 mol) in 30 ml of methanol, 10% Pd/C (150 mg) was added. The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (XII) (96% yield).
4. The compound (XII) was protected using suitable protecting agent to give protected product of (XIII). The compound XII (100 grams) was added to a mixture of chloroform (900 mL), imidazole (77.57 grams) and tert-butyldimethylsilyl chloride (TBDMS-Cl) solution at 25-30° C., and was refluxed for 10 hours. The progress of the reaction was monitored by HPLC. After the completion of the reaction, the reaction mass was cooled to 15-20° C., washed with 5% HCl solution and distilled under vacuum at 45-50° C. to obtain a residue. The residue was mixed with cyclohexane (500 mL) and stirred for 1 hour at 25-30° C. The resultant solid was filtered, washed with cyclohexane and dried at 60-65° C. for 5 hours to get the protected compound XIII (85% yield).

### Example 3: Preparation of Navafenterol by Route 1 Synthesis from its Intermediates of the Formula (XIII) and Formula (XVI)

1. A solution of (XIV) (10.0 g, 48.6 mmol) in 250 ml of toluene was heated until a temperature between 65° C. and 70° C. was reached and sodium methoxide (1.05 g, 19.4 mmol) was added to the reaction mass. A solution of methyl di (2-thienyl) glycolate (MDTG-XV) (12.09 g; 47.15 mmol) in 60 ml of toluene was added under a flow of nitrogen at a temperature between 75° C. and 85° C. during around 1 hour. At a temperature between 75° C. and 85° C. the azeotrope of toluene and methanol was removed under a flow of nitrogen and by distillation (with replacement of fresh toluene) until the reaction is considered complete. The reaction mixture was washed 3 times with a 20% aqueous solution of NaCl. The organic phase was dried and concentrated under vacuum at a temperature up to 40° C. until a final volume of the reaction mass reached to 125 ml. The suspension was cooled to 10°-15° C. and stirred at this temperature for at least 4 hours. The obtained product was filtered and washed with 50 ml of chilled toluene and dried at 60° C. to a constant weight to get product (XVI).
2. A solution of product (XIII) (20 g) in 140 ml Toluene was added with TEA (1.2 eq.) at below 10° C. A solution of (XVI) in 60 ml Toluene was added to the reaction mixture at the same temperature. The temperature was raised to room temperature and the reaction mass was heated at 60° C. The reaction mixture was maintained at the same temperature till reaction completion. Water was added to the reaction mass and the layers were separated. The toluene layer was washed using 10% aq. HCl solution, twice with water followed by brine solution. The organic layer is dried using anhydrous Na₂SO₄ and the solvent was distilled under vacuum until a final volume of the reaction mass reached to 125 ml. The suspension was cooled to 10°-15° C. and stirred at this temperature, at least, 4 hours. The obtained product was filtered and washed with 50 ml of chilled toluene. The product was dried at 60° C.to a constant weight to get product (I).

### Example 4: Preparation of Navafenterol by Route 2 Synthesis from its Intermediates of the Formula (XVIII) and Formula (XV)

1. A solution of product (XIII) (20 g) in 140 ml DCM was added with TEA (1.2 eq.) at below 10° C. A solution of (XVII) in 60 ml DCM was added to the reaction mixture at the same temperature. The temperature was raised to room temperature. The reaction mixture was maintained at the same temperature till reaction completion. Water was added to the reaction mass and the layers were separated and the DCM layer was washed twice with water followed by brine solution. The organic layer was dried using anhydrous Na₂SO₄. The solvent was distilled under vacuum to get the compound (XVIII).
2. A solution of Product (XVIII) (50.0 g, 96.04 mmol) in 500 ml of toluene was heated until a temperature between 65° C. and 70° C. was reached and sodium methoxide (2.08 g, 38.5 mmol) was added to the reaction mass. A solution of methyl di (2-thienyl) glycolate (MDTG-(XV) (23.69 g; 93.16 mmol) in 100 ml of toluene was added under a flow of nitrogen at a temperature between 75° C. and 85° C. during around 1 hour. At a temperature between 75° C. and 85° C. the azeotrope of toluene and methanol was removed under a flow of nitrogen and by distillation (with replacement of fresh toluene) until the reaction was considered complete. The reaction mixture was washed 3 times with a 20% aqueous solution of NaCl. The organic phase was dried and concentrated under vacuum at a temperature up to 40° C. until a final volume of the reaction mass reached to 125 ml. The suspension was cooled to 10°-15° C. and stirred at this temperature, at least, 4 hours. The obtained product was filtered and washed with 50 ml of chilled toluene. The product was dried at 60° C. to a constant weight to get product (I).

### Example 5: Preparation of Navafenterol by Route 3 Synthesis from its Intermediates of the Formula (XIa) and Formula (XVI)

1. A stirred solution of (VII) in THF was cooled to 10-15° C. and aq.HBr (48% 1.1 m.eq) was added. The resulting mixture was stirred at the same temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate was distilled under vacuum at below 45° C. to get the compound bromo hydrin. The obtained bromo hydrin is dissolved in chloroform 250 ml, added 4.0 eq of imidazole, stirred the reaction mixture at room temperature. Added a mixture solution of TBDMS in chloroform at below 25° C. Heated the reaction mixture for 4-6 hours for reaction completion. After reaction completion, reaction mass was cooled and water 250 ml was added. Stirred the mass for 15 min at RT, separated the layers, washed the organic layer using saturated brine and dried over anhydrous Na₂SO₄. Distilled off solvent under vacuum, added 250 ml of cyclohexane, cooled to 10° C. Stirred and filtered, washed with cyclohexane 50 ml. Dried the product to get 85% of the title compound VIIIa.
2. To a stirred solution of THF (25 mL) and bromo hydrin (VIIIa) (2.5 g, 0.051 mol) a solution of compound (IXa) (1.22 g, 0.0051 mol) in THF 25 ml was added in a dropwise manner at room temperature under nitrogen. After complete addition, the mixture was allowed to warm to 50° C. and stirred for 4 h. The resulting homogeneous mixture was quenched with saturated aqueous Na₂SO₃ and extracted with CH₂Cl₂ and purified by flash chromatography on silica gel using a gradient mixture of ethyl acetate-n-hexane to give the compound (XIX) (78% yield) as a solid.
3. A solution of product (XIa) (20 g) in 200 ml Toluene was added with TEA (1.5 eq.) at below10° C. A solution of (XVI) in 60 ml Toluene was added to the reaction mixture at the same temperature. The temperature was raised to room temperature and the reaction mass was heated at 60° C. The reaction mixture was maintained at the same temperature till reaction completion. Water was added to the reaction mass and the layers were separated. The toluene layer was washed using 10% aq. HCl solution, twice with water followed by brine solution. The organic layer is dried using anhydrous Na₂SO₄ and the solvent was distilled under vacuum until a final volume of the reaction mass reached to ˜100 ml. The suspension was cooled to 10°-15° C. and stirred at this temperature, at least, 4 hours. The obtained product was filtered and washed with chilled toluene twice with 25 ml. The product was dried at 60° C. to a constant weight to get product (XIX) with 85% yield.
4. To the above compound (XIX) (2.5 g, 0.0024 mol) in 30 ml of methanol, 1% Conc.HCl, 10% Pd/C (250 mg) was added. The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (I) (96% yield).

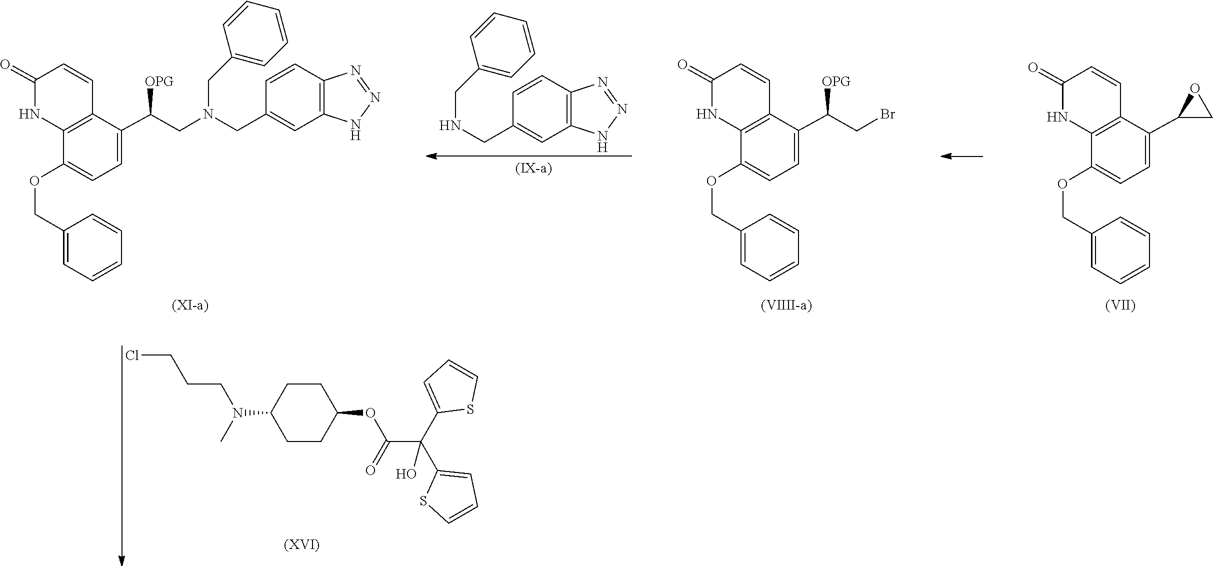

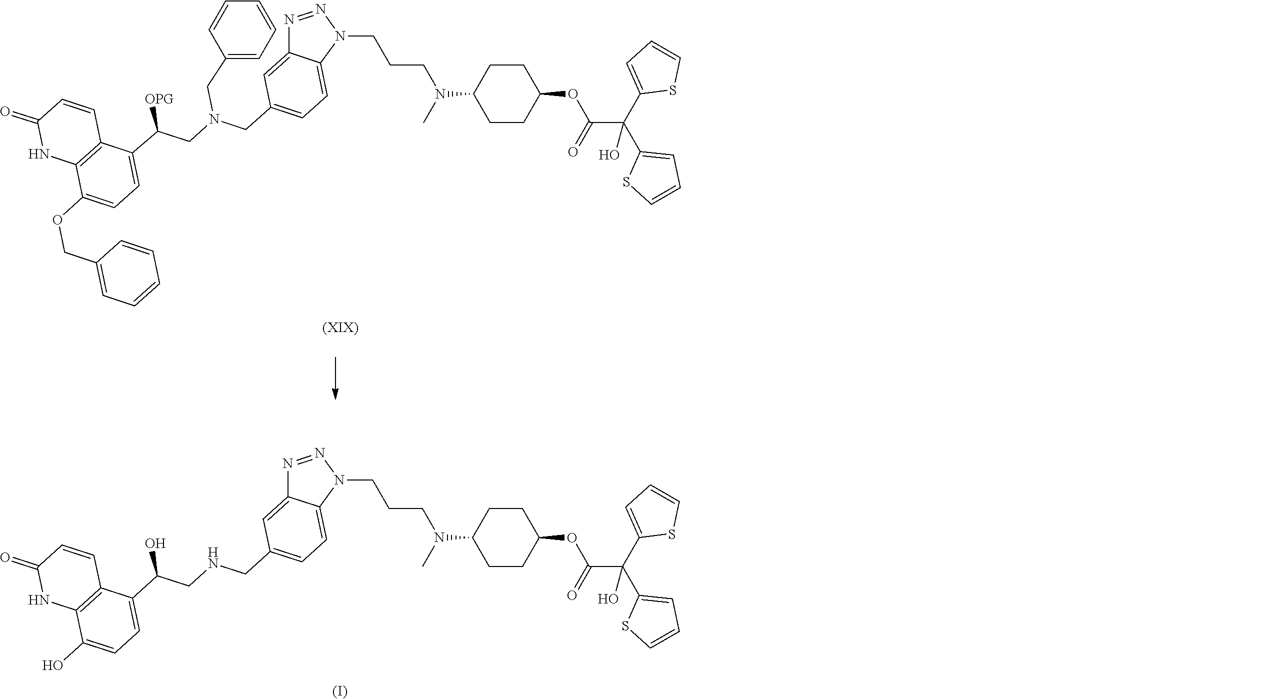

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the spirit or scope of the present invention.

1) A process for preparation of Navafenterol compound having the Formula (I) 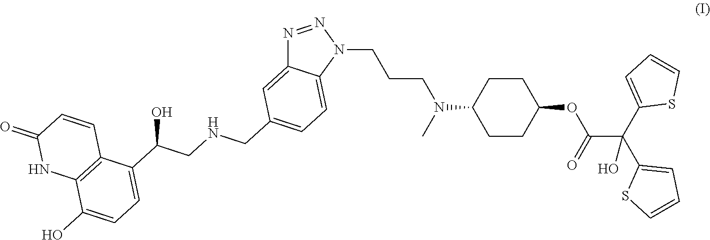 [SMILES: CN(CCCn1nnc2cc(CNC[C@H](O)c3ccc(O)c4[nH]c(=O)ccc34)ccc21)[C@H]1CC[C@H](OC(=O)C(O)(c2cccs2)c2cccs2)CC1]
comprising the steps of:
a) addition of the compound of Formula (II) to a solvent followed by adding a base and a benzylating agent to obtain a compound of Formula (III); 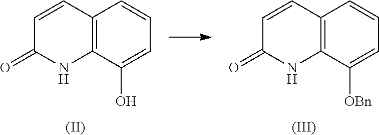 [SMILES: O=c1ccc2cccc(O)c2[nH]1 ; O=c1ccc2cccc(OCc3ccccc3)c2[nH]1 ; "(II)" ; "(III)"]
b) addition of the compound of Formula (III) to an acid and a brominating agent to make a reaction mixture; to obtain a compound of Formula (IV); 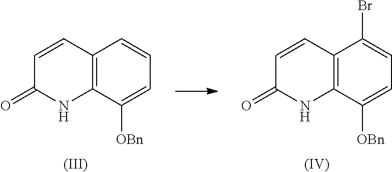 [SMILES: O=c1ccc2cccc(OCc3ccccc3)c2[nH]1 ; O=c1ccc2c(Br)ccc(OCc3ccccc3)c2[nH]1 ; "(III)" ; "(IV)"]
c) carbonylation of compound of formula IV with strong base to obtain compound of formula V; 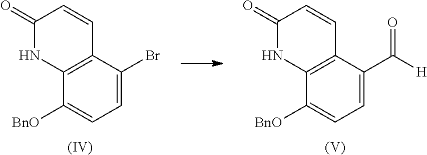
d) chiral epoxidation of compound of formula V using chiral sulphide (VI), in presence of base to obtain compound of formula VII; 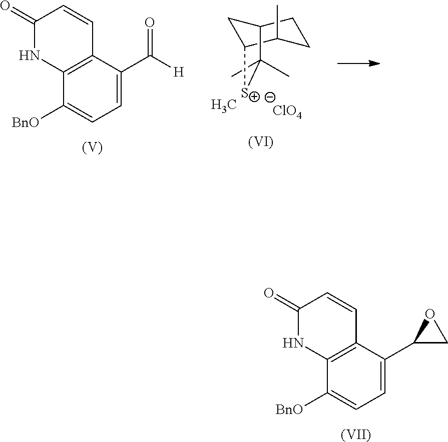
e) cleavage of chiral epoxide compound of formula VII using brominating agent to obtain compound of formula VIII; 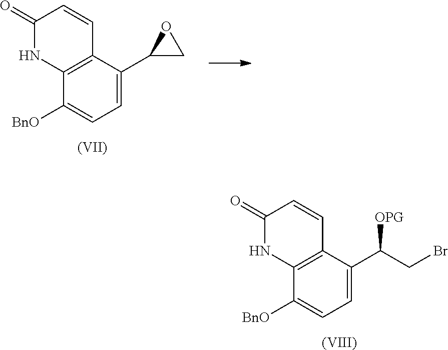
f) treatment of compound of formula VIII with compound of formula IX in base to obtain compound of formula XI; 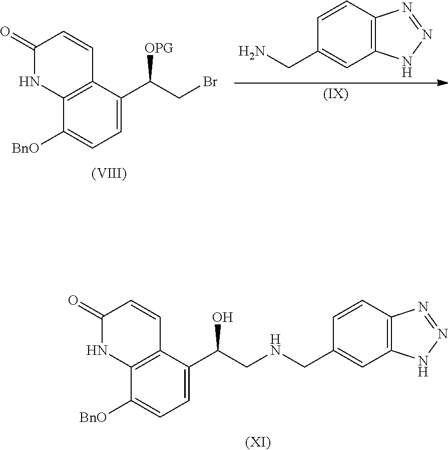
g) hydrogenation of compound of the Formula XI in presence of catalyst to obtain compound of Formula XII, 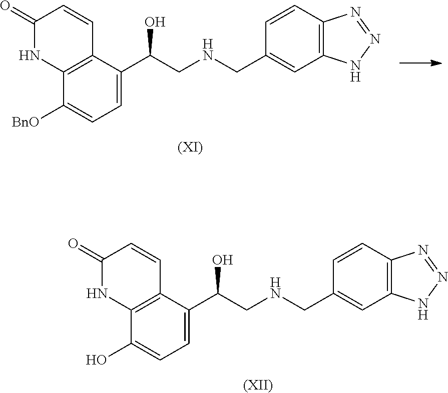
h) protection of compound of Formula XII, with a protecting agent to obtain compound of Formula XIII; 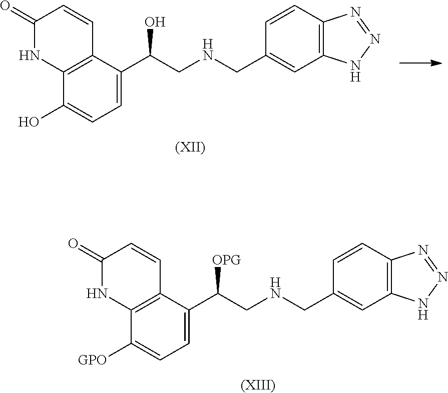
i) synthesis of Navafenterol having the Formula (I) from the compound of Formula (XIII)
(1) by condensation with the compound of Formula (XVI); or
(2) via intermediates of the compound of Formula (XVIII) and compound of Formula (XV); or
(3) via intermediates of the compound of Formula (XI-a) and compound of Formula (XVI).

2) A process as claimed in claim 1, wherein synthesis of Navafenterol from the intermediate compound of Formula (XIII) and compound of Formula (XVI) includes the following steps:
a) condensation of compound of Formula XIV by adding a solution of compound of Formula XV to obtain compound of Formula XVI; and 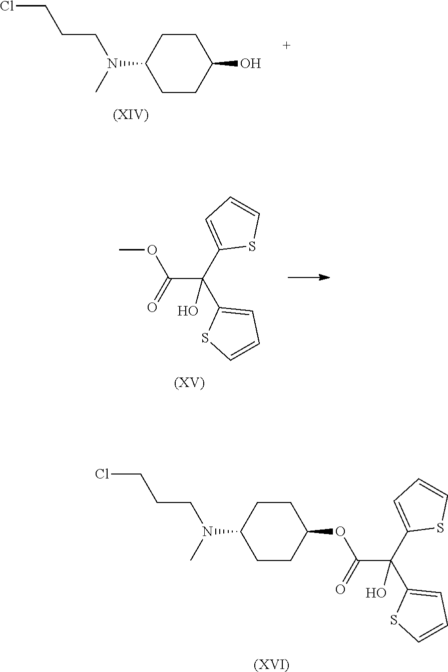
b) condensation of compound of formula XIII and compound of formula XVI in presence of base to obtain Navafenterol of Formula I.

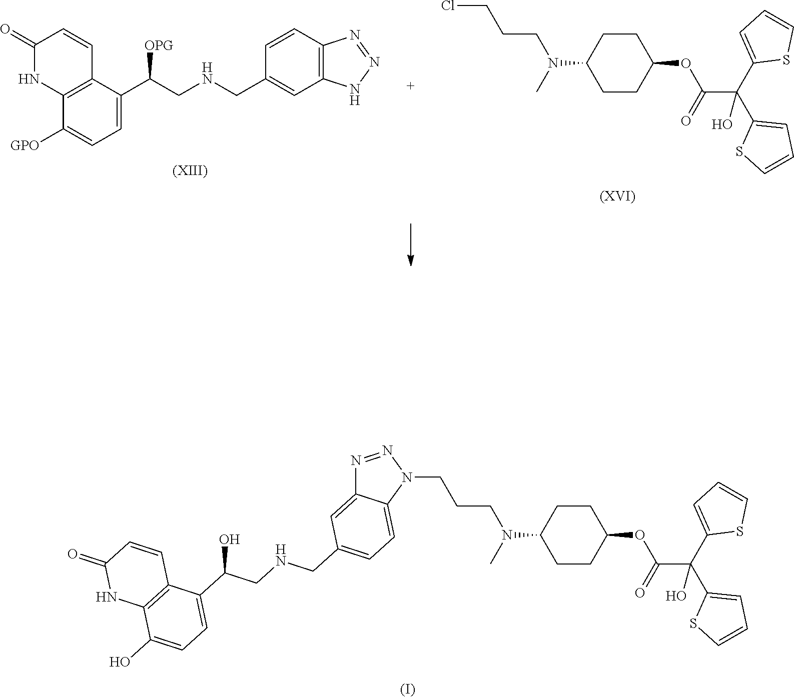

3. A process as claimed in claim 1, wherein synthesis of Navafenterol from the intermediate compound of Formula (XIII) via intermediates of the compound of Formula (XVIII) and compound of Formula (XV) includes the following steps:
a) condensation of compound of Formula (XIII) with compound of Formula (XVII) to give compound of Formula (XVIII). 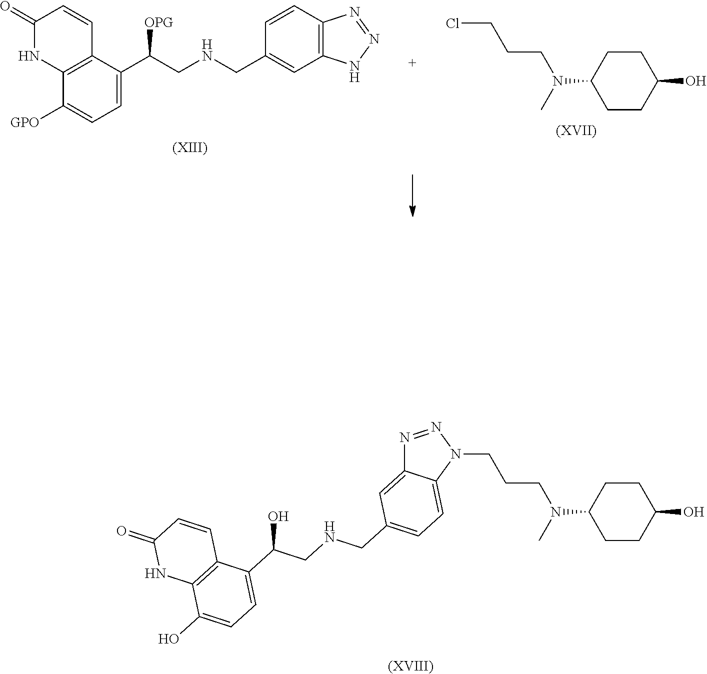
b) condensation of compound of Formula (XVIII) with compound of Formula (XV) to give compound of Formula (I).

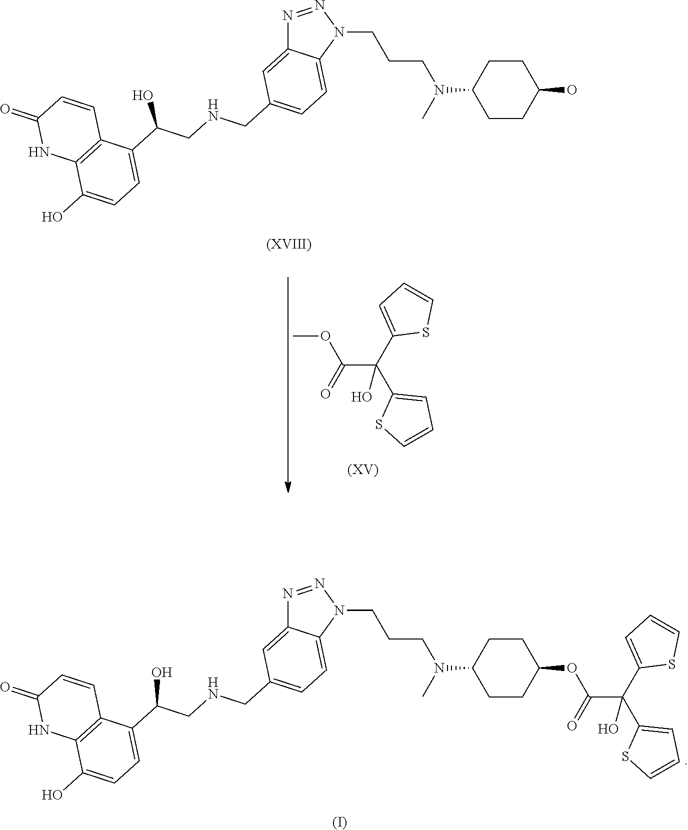

4. A process as claimed in claim 1, wherein synthesis of Navafenterol from the intermediate compound of Formula (XIII) via intermediates of the compound of Formula (XI-a) and compound of Formula (XVI) includes the following steps:
a) protection of compound of formula VIII with protecting agents to obtain compound of formula VIII(a); 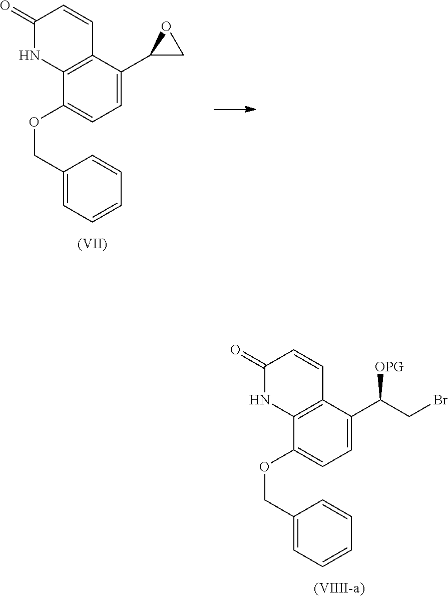
b) treatment of compound of formula VIII(a) with compound of formula IX(a) to obtain compound of formula XI(a); 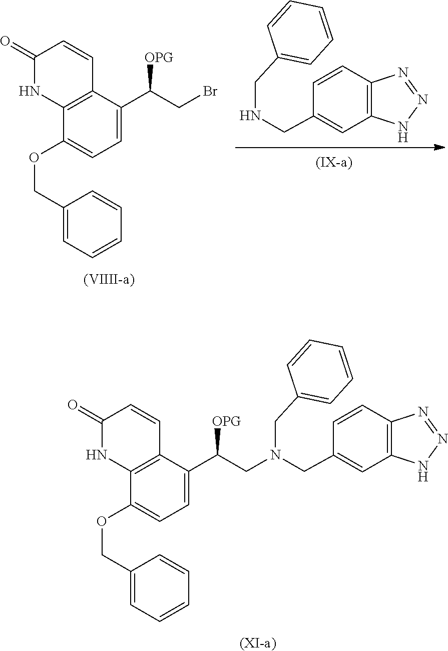
c) condensation of compound of Formula XI(a) and compound of Formula XVI to obtain compound of formula XIX; 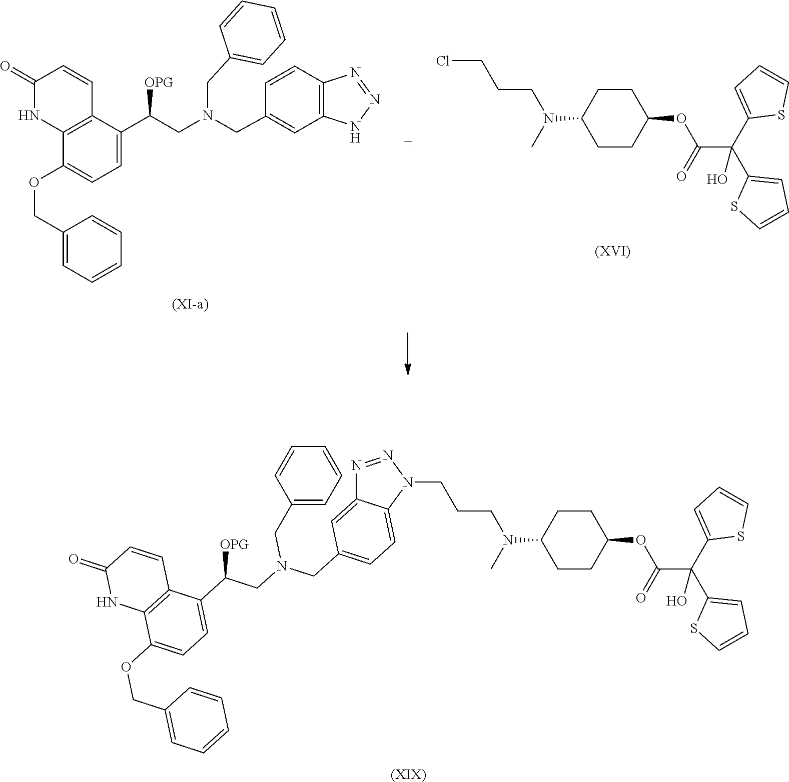
d) hydrogenation of compound of formula XIX to obtain compound of formula I.

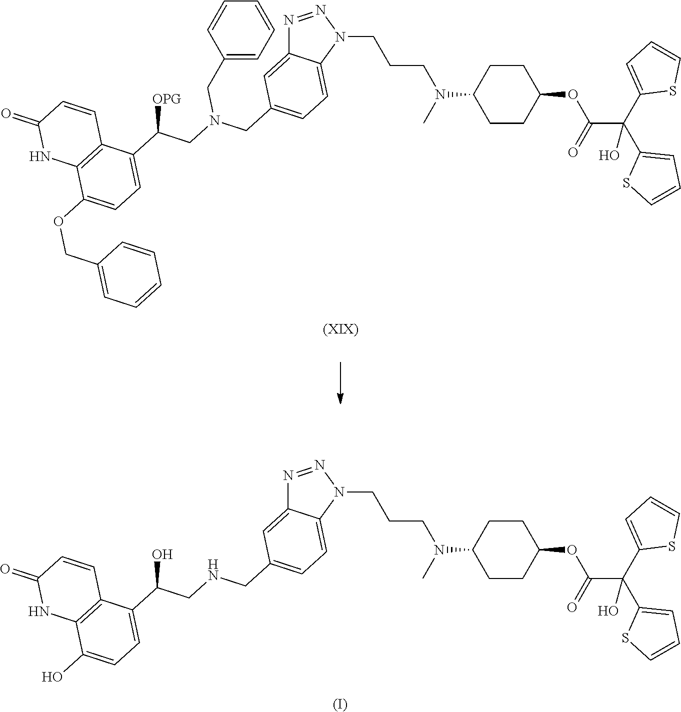

5. The process as claimed in claim 1, for the preparation of the intermediate compound of Formula XIII wherein:
a) addition of compound of Formula II is carried out in presence of solvents selected from acetone, THF or DMF and base selected from group of K₂CO₃, Na₂CO₃, or Cs₂CO₃; followed by addition of benzylating agent selected from benzyl bromide, benzyl chloride or benzyl iodide to obtain compound of formula III;
b) addition of compound of Formula III is carried out in halosolvents, acetic acid, formic acid, hydrobromic acid; further bromination with bromine, 1,3-dibromohydantoin, tetra butyl ammonium tribromide, NBS to obtain compound of formula IV;
c) carbonylation of compound of formula IV is carried out in presence of solvents selected from tetrahydrofuran, methyl tert-butylether, diisopropyl ether, or diethyl ether, followed by treating with strong base selected from n-butyl lithium, s-butyl lithium, lithium diisopropylamide, potassium bis(trimethylsilyl)amide, in turn addition of N,N-dimethylformamide and tetrahydrofuran in the ratio of 1:1 to obtain compound of formula V;
d) chiral epoxidation of compound of formula V is carried out using chiral sulphide derivative (VI), in presence of base selected from potassium hydroxide, sodium hydroxide, lithium hydroxide, in tert-butyl alcohol, isopropyl alcohol, methanol to obtain compound of formula VII;
e) cleavage of chiral epoxide compound of the formula VII is carried out in solvents selected from Tetrahydrofuran, or halo solvents, and the brominating agent selected from bromine, 1,3-dibromohydantoin, NBS to obtain compound of formula VIII;
f) treatment of compound of formula VIII with compound of formula IX is carried out in solvents selected from acetone, THF, DMF using base selected from K₂CO₃, Na₂CO₃, Cs₂CO₃, to obtain compound of formula XI;
g) hydrogenation of compound of formula XI is carried out in methanol, ethanol, Isopropyl alcohol, ethyl acetate, in presence of catalyst to obtain compound of formula XII;
h) protection of compound of formula XII is carried out with protecting agents selected from THP, TBDMS, TMS and benzyl bromide, benzyl chloride, benzyl iodide, in solvents selected from acetone, THF, DMF, acetonitrile, 2-metyl THF, MIBK, halo solvents using base selected from K₂CO₃, Na₂CO₃, Cs₂CO₃, imidazole to obtain formula XIII.

6. The process as claimed in claim 5, for the preparation of the intermediate compound of Formula XIII wherein the addition of benzylating agent is carried out at the temperature of about 0° C. to 5° C.; carbonylation of compound of formula IV is carried out at the temperature of about 0° C. to −78° C. for 2 to 3 hours, and cleavage of chiral epoxide of formula VII is carried out at the temperature of about 10° C.-15° C.; hydrogenation is carried out in presence of catalyst selected from the group of Pd/C, Pd/BaSO₄, or Raney nickel.

7. The process as claimed in claim 2, for the preparation of Navafenterol of the Formula I from the compound of Formula (XIII) by condensation with the compound of Formula (XVI); wherein the condensation of Formula XIV is carried out in solvent toluene using base sodium methoxide followed by adding a solution of compound of Formula XV in toluene to obtain compound of Formula (XVI); isolation of compound of formula XVI is carried out in toluene under cooling; condensation of compound of Formula XIII and compound of Formula XVI is carried out in presence of base TEA in toluene; the isolation of formula I in toluene is carried out under cooling.

8. The process as claimed in claim 7, for the preparation of Navafenterol of the Formula I from the compound of Formula (XIII) by condensation with the compound of Formula (XVI), wherein the condensation of compound of Formula XIII and compound of Formula XVI is carried out at the temperature at below 10° C.-15° C. for at least 4 hours; the condensation of compound of Formula XVIII in solvent toluene using base sodium methoxide is carried out at the temperature at about 75° C. and 85° C.

9. The process as claimed in claim 3, for the preparation of Navafenterol of the Formula I from the compound of Formula (XIII) via intermediates of the compound of Formula (XVIII) and compound of Formula (XV) wherein reaction of compound of Formula XIII with compound of Formula XVII in presence of base TEA is carried out under cooling in dichloromethane to obtain compound of XVIII; isolation of compound of formula XVIII as residue; condensation of compound of Formula XVIII is carried out in solvent toluene using base sodium methoxide followed by adding a solution of Formula XV in toluene to obtain compound of Formula I; isolation of compound of formula I is carried out in toluene under cooling.

10. The process as claimed in claim 4, for the preparation of Navafenterol of the Formula I from the compound of Formula (XIII) via intermediates of the compound of Formula (XI-a) and compound of Formula (XVI), wherein protection of compound of formula VIII is carried out with protecting agents selected from THP, TBDMS, TMS and benzyl bromide, benzyl chloride, benzyl iodide, in solvents selected from acetone, THF, DMF, acetonitrile, 2-metyl THF, MIBK, using base selected from K₂CO₃, Na₂CO₃, Cs₂CO₃, imidazole to obtain compound of formula VIII(a); treatment of compound of formula VIII(a) with compound of formula IX (a) in carried out in solvents selected from acetone, THF, DMF using base selected from K₂CO₃, Na₂CO₃, Cs₂CO₃, to obtain compound of formula XI(a); condensation of compound of Formula XI (a) and compound of Formula XVI is carried out in presence of base TEA in toluene, and isolation is carried out in toluene under cooling to obtain compound of formula XIX; hydrogenation of compound of formula XIX is carried out in acidic medium using solvents selected from methanol, ethanol, Isopropyl alcohol, ethyl acetate, in presence of catalyst selected from the group of Pd/C, Pd/BaSO₄, or Raney nickel to obtain compound of formula I.

11. The process as claimed in claim 4, wherein the intermediate compound of formula XIX is utilized for the synthesis of Navafenterol of formula I. [chemical structure]
